(19) United States
(12) Patent Application Publication
(10) Pub. No.: US 2010/0111755 A1
(43) Pub. Date: May 6, 2010

(54) AIR TREATMENT DEVICE UTILIZING A SENSOR FOR ACTIVATION AND OPERATION

(75) Inventors: Jeffrey Bankers, Phoenix, AZ (US); Sanam Nassirpour, Phoenix, AZ (US); Kevin Hafer, Chandler, AZ (US)

Correspondence Address: SNELL & WILMER, LLP (Dial Corp), ONE ARIZONA CENTER, 400 E. VAN BUREN, PHOENIZ, AZ, 85004-2202, US

(73) Assignee: THE DIAL CORPORATION, Scottsdale, AZ (US)

(21) Appl. No.: 12/595,607

(22) PCT Filed: Apr. 11, 2008
(86) PCT No.: PCT/US08/59989

§ 371 Date: Oct. 12, 2009

Related U.S. Application Data
(60) Provisional application No. 60/911,762, filed on Apr. 13, 2007.

(51) Int. Cl. A61L 9/00

(52) U.S. Cl. 422/3; 422/116; 422/111; 422/4

(57) ABSTRACT

The present invention provides a system for detecting a malodor and dispensing an air treatment when needed. In an exemplary embodiment of the present invention, the system includes a selection mechanism which allows the user to choose how the system dispenses the air treatment, a sensor, a timing control circuit coupled with the sensor, and a delivery system coupled with the timing control circuit. Upon detection of a malodor by the sensor, the sensor communicates with the timing control circuit which, in turn, communicates with the delivery system to dispense the air treatment. In another exemplary embodiment of the present invention, an air-freshening system is shown that combines a continuous dispensing system with a sensing dispensing system.

### FIELD OF INVENTION

The present invention generally relates to vapor-dispensing devices. More particularly, the system may include active vapor-dispensing, based in part upon a sensor configured to detect different malodors and a timing control circuit configured to operate in different modes.

### BACKGROUND OF THE INVENTION

Vapor-dispensing products typically include a volatizable material and a transport system configured to facilitate evaporation of the volatizable material into the surrounding air. For example, in some systems, a liquid material is contained in a reservoir bottle, and is volatized with heat, pump, aerosol, fan, etc.

Known vapor-dispensing devices of this type may be improved upon in a number of respects. For example, some vapor-dispensing devices dispense fragrance vapor at times when the fragrance may not be needed. Furthermore, some devices do not emit enough fragrance vapors when certain malodors are present. Additionally, certain malodors associated with different locations, i.e., kitchen, bathroom, laundry room, locker, children's closet, etc, may be treated more effectively with certain fragrances.

### SUMMARY OF THE INVENTION

The present invention provides a system for dispensing an air treatment when needed. In an exemplary embodiment of the present invention, an air treatment system for detecting a malodor and dispensing an air treatment is provided. In accordance with various embodiments, the system includes a housing, a sensor, a timing control circuit coupled with the sensor, and a delivery system coupled with the timing control circuit. Upon detection of a malodor by the sensor, the sensor communicates with the timing control circuit which, in turn, communicates with the delivery system to dispense the air treatment. The system may also include a selection mechanism which allows the user to choose how the system dispenses the air treatment. In an exemplary embodiment, the device is further configured to detect the air treatment or fragrance and regulate later dispensing accordingly.

### BRIEF DESCRIPTION OF THE DRAWINGS

These and other features, aspects and advantages of the present invention will become better understood with reference to the following description, appending claims, and accompanying drawings where:

FIG. 1 shows a block diagram of a method and system of an exemplary air treatment device;

FIG. 2 shows a flow diagram of a method and system of active dispensing, according to an exemplary embodiment; and

FIG. 3 shows a flow diagram of a method and system of active dispensing, according to an exemplary embodiment.

### DETAILED DESCRIPTION

The following descriptions are of exemplary embodiments of the invention only, and are not intended to limit the scope or applicability of the invention in any way. Rather, the following description is intended to provide convenient illustrations for implementing various embodiments of the invention. As will become apparent, various changes may be made without departing from the spirit or scope of the invention as set forth in the appended claims.

For the sake of brevity, functional embodiments of the apparatus and systems (and components of the individual operating components of the systems) may not be described in detail herein. Furthermore, the connecting lines shown in the various figures contained herein are intended to represent exemplary functional relationships or physical connections between the various elements. It should be noted that many alternative or additional functional relationships or physical connections may be present in a practical system.

In accordance with various embodiments of the present invention, an air treatment device is configured to detect a malodor with a sensor and actively respond to the malodor by dispensing an air treatment or fragrance into the surrounding environment. In an exemplary embodiment, the device is further configured to detect the air treatment or fragrance and regulate later dispensing accordingly.

In accordance with various embodiments of the present invention, activation or operation of an air treatment device with a sensor depends on the consumer's desired interaction (or lack thereof). Specifically, in certain instances a consumer may wish to have timed treatment, i.e., the air treatment device is active at certain times or for certain periods. Additionally or alternatively, in certain situations a consumer may wish to have a sensor activated or deactivated air treatment device, for example, for conservation of power. Still further, a consumer may wish to have both timed treatment and sensor activated treatment.

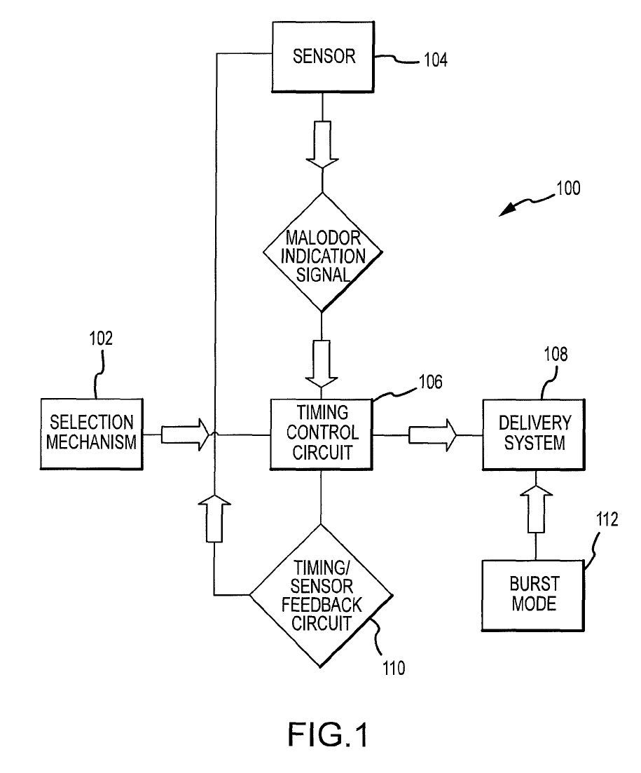

Systems and methods are described below wherein the combination of the consumer's interaction, the timed treatment of the air, and the sensor activated treatment of the air, are interdependent. In an exemplary embodiment, for example, the one shown in FIG. 1, an air treatment system 100 includes a housing, a selection mechanism 102, a sensor 104, a timing control circuit 106, and a delivery system 108.

The selection mechanism 102 operates to indicate in what mode the air treatment system will operate. In accordance with various embodiments, the selection mechanism 102 is set for a timed treatment, a sensor activated treatment, or a combination thereof. In such embodiments, each of the timed and sensor activated treatment may be adjusted to high, medium, low, or off, depending on the consumer's desired level of treatment. The selection mechanism 102 communicates with the timing control circuit 106 to help assure the consumer's desired level of treatment. In accordance with one aspect of various embodiments, the selection mechanism 102 is a switch or any other suitable mechanism that allows the consumer to indicate in what mode the air treatment system will operate.

In exemplary embodiments, the sensor 104 samples the environment for either the level of active air treatment material or the quality of the current environment. The sensor 104 is capable of distinguishing between a plurality of malodors in a plurality of locations such that a single air treatment system may be used in multiple locations. In an exemplary embodiment, the sensor 104 is a metal oxide sensor (MOS), however, it should be understood by one of ordinary skill in the art that any suitable sensor exhibiting the qualities discussed herein may be used in the context of the present invention.

The timing control circuit 106 activates the air treatment system 100 and causes the delivery system 108 to disperse the air treatment. The timing control circuit 106 allows for a timed treatment, a sensor activated treatment or a combination thereof. For the timed treatment, the air treatment system 100 is activated in a timed manner that is controlled in terms of both the duration of the treatment, commonly referred to as “on time” and the intervening time between treatments, commonly referred to as “off time”. In an exemplary embodiment, the timing control circuit 106 contains interval timed programming wherein air treatment material is dispersed in predetermined intervals during the timed treatment.

For the sensor activated treatment, the timing control circuit 106 and the sensor 104 are interconnected by a feedback circuit 110 such that the sensor 104 controls the timing control circuit 106 and vice-versa. Thus, the sensor 104 controls the timing control circuit 106 in that, when the sensor 104 detects a malodor, an indication signal is sent to the timing control circuit 106. The timing control circuit 106 will then cause the delivery system 108 to disperse the air treatment.

In an exemplary embodiment, the timing control circuit 106 disengages the sensor 104 for an operational time period via the feedback circuit 110 if the timing control circuit 106 determines a significant time period of increased air treatment.

In another exemplary embodiment, the device is further configured to detect the air treatment or fragrance and regulate later dispensing accordingly. In accordance with an aspect of an exemplary embodiment, the timing control circuit 106 disengages the sensor 104 for an operational time period via the feedback circuit 110 if the sensor 104 determines a significantly increased air treatment intensity. In accordance with another aspect of an exemplary embodiment, the timing control circuit 106 engages the sensor 104 for an operational time period via the feedback circuit 110 if the sensor 104 determines a significantly decreased air treatment intensity.

Among other advantages, in some embodiments the interconnection of the timing control circuit 106 and the sensor 104 by the feedback circuit 110 allows for power conservation. For example, in exemplary embodiments the sensor 104 has to be heated to work and power is conserved if the timing control circuit 106 also controls the sensor 104. For example, the timing control circuit 106 can turn off the sensor 104 when it is not needed.

The delivery system 108 expels the air treatment material when the level or presence of a malodor is detected by the sensor 104 or in accordance with a timed treatment. The delivery system 108 receives the signal from the timing control circuit 106. The delivery system 108 can be any system capable of dispensing the air treatment material as appreciated by one of ordinary skill in the art.

Another aspect in accordance with various embodiments of the invention is the burst mode 112. In one embodiment, the burst mode 112 is initiated by an actuator on the device to immediately dispense the air treatment. In one embodiment, the burst mode 112 would communicate with the timing control circuit 106, which would then activate the delivery system 108 to relatively immediately dispense the air treatment. In another embodiment, as shown in FIG. 1, the burst mode 112 would communicate directly with the delivery system 108 to relatively immediately dispense the air treatment. A user may actuate a button or another indication may be provided, which would signal that immediate dispensing of an air treatment is desired. This may further provide an air treatment to mask, neutralize or otherwise remove malodors within the area. The burst mode may function alone or in combination with either or both of a timed treatment and a sensor activated treatment.

Another aspect in accordance with various embodiments of the invention is a “kill mode.” A kill mode turns off the air treatment device if a determination is made the device it is not positioned upright. In an exemplary embodiment, the air treatment device comprises an actuator configured to be pushed in by a surface, upon which the device is situated, when the device is upright, and not pushed in when the device is positioned other than upright. In another exemplary embodiment, the air treatment device comprises an acrylic level vial, in communication with a circuit or signal pathway within the air treatment device, configured to determine the orientation of the device and take appropriate action in response thereto. In yet another embodiment, the air treatment device is mechanically or electrically configured such that a circuit or signal is disrupted or stopped if the device is positioned other than upright. In general, any configuration for turning off the air treatment device if a determination is made the device it is not positioned upright is contemplated for use in the present invention.

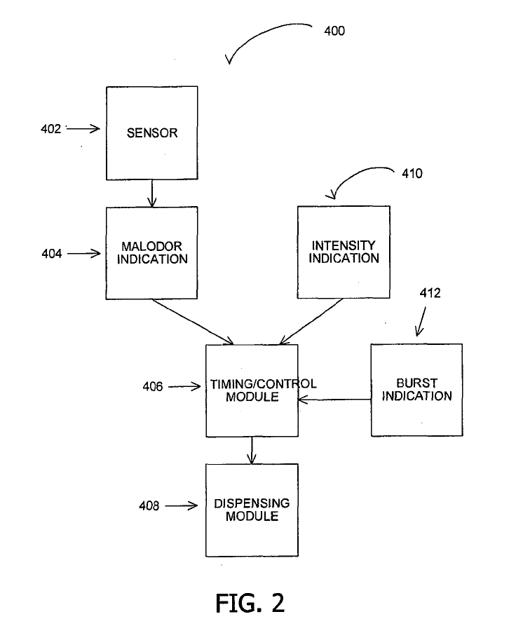

FIG. 2 is a flow diagram of a method and system 400 of active dispensing of an air treatment, according to an exemplary embodiment. In various exemplary embodiments, method and system 400 dispense an air treatment in many different manners, such as the three shown in FIG. 2. In accordance with one exemplary embodiment, system 400 dispenses an air treatment includes a sensor 402 sensing an activation malodor and sending a malodor indication 404 to a timing/control module 406. Any suitable sensor, including those discussed above, exhibiting the qualities discussed herein may be used. The timing/control module 406 would then send a dispensing signal to a dispensing module 408 to indicate to the dispensing module 408 to dispense a volatizable liquid or fragrance. This is one mode of operation for system 400.

In accordance with another exemplary embodiment, system 400 includes the setting of an intensity indication 410. The intensity indication 410 may be set by a user, which would send an activation signal to the timing/control module 406 to periodically activate the dispensing module 408 and at a desired intensity. This may be a user preference, in that a volatizable liquid or fragrance would be dispensed at regular periodic intervals and at a desired intensity, both of which may be increased or decreased to certain limits, depending on the consumer's desired treatment intervals and intensity. In accordance with one aspect of an exemplary embodiment, this is a factory setting, pre-set before the system is activated.

In accordance with yet another exemplary embodiment, system 400 includes a burst indication 412. The burst indication 412 may be initiated by an actuator on the system to immediately dispense the air treatment. The burst indication 412 would communicate with the timing/control module 406, which would then activate the dispensing module 408 to relatively immediately dispense the air treatment. In a particular embodiment, a user actuates a button or another indication is provided, which signals that immediate dispensing of an air treatment is desired. This may further provide an air treatment to mask, neutralize or otherwise remove malodors within the area.

In addition to dispensing an air treatment and in accordance with various embodiments, dispensing module 408 also comprises an aerosol or other method of rapid deploying an air treatment within the area. For example, system 400 may include a multi-position switch to indicate to the system which mode to operate in. Importantly, each mode may function alone or in combination with one or more of a sensor mode, an intensity indication mode and a burst indication mode.

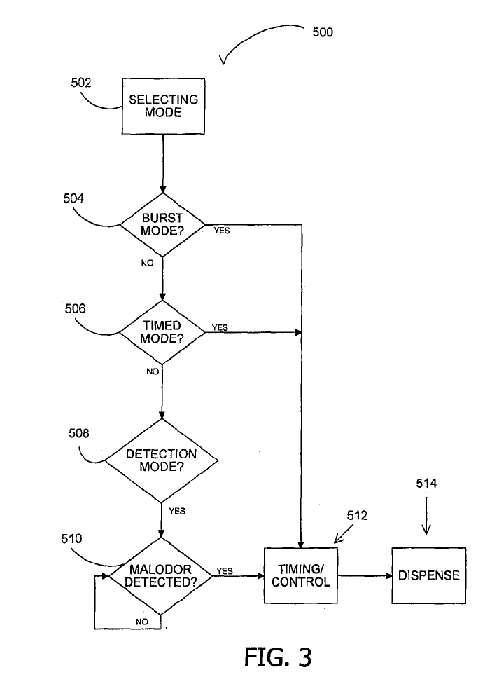

FIG. 3 shows a flow diagram of a method 500 of active dispensing, according to an exemplary embodiment. In this embodiment, method 500 includes selecting a mode 502, determining if it is a burst mode 504, determining if it is a timed mode 506, determining if it is a detection mode 508, malodor detection 510, performing timing and control 512, and dispensing 514.

Selecting a mode 502, in an embodiment, includes selecting a mode of operation of an air treatment device. This may be done manually by a user, or may be generally automatic. The device may comprise any number of modes, from one to hundreds or more. Each mode may function alone or in combination with another or others. In this embodiment there are three modes.

After selecting a mode 502, a determination is made of whether the system is in a burst mode 504. If the system is in a burst mode 504, the YES leg is taken to the timing and control step 512 followed by the dispensing step 514, which would, relatively immediately, dispense an air treatment in a burst or aerosol configuration. In an exemplary embodiment, in a burst mode 504, the consumer may activate the burst mode 504, relatively independent of a timed mode 506 or a detection mode 508. Selecting burst mode 504 may or may not reset the timed mode 506 or deactivate the detection mode 508 for a time period, depending on the air treatment and its method of delivery. If the burst mode determination is negative, the NO leg is taken to determine whether the system is in a timed mode 506.

If the system is in a timed mode 506, the YES leg is taken to the timing and control step 512 followed by the dispensing step 514. In an exemplary embodiment of a system in a timed mode 506, the timing and control step 512 periodically sends a signal to the dispensing step 514. This periodic signaling may be preset or may be customizable by the user in terms of duration as well as intensity. If the timed mode determination is negative, the NO leg is taken to determine whether the system is in a detection mode 508.

If the system is in a detection mode 508, the YES leg is taken to the malodor detection step 510. If a particular type of malodor is detected 510, the YES leg is taken to the timing and control step 512 followed by the dispensing step 514. If the system is in a detection mode 508 and a malodor is not detected 510, the NO leg is taken and the malodor detection step 510 continues until a malodor is detected 510, upon which the YES leg is taken to the timing and control step 512 followed by the dispensing step 514.

In an exemplary embodiment, upon activation (or at any point during operation) the consumer sets the mode in which the air treatment system will operate via the selection mechanism. Next, the timing control circuit activates the air treatment device according to the selection mechanism. The selection mechanism controls the duration of the treatment, “on time” and the intervening time between treatments, “off time” thereby allowing the delivery system to dispense air treatment according to the desired level chosen by the consumer.

Additionally, during operation of a sensing treatment, the sensor and its control circuitry may also continuously sample the environment for either the level of active air treatment material or the environmental air quality. The level of active air treatment material is sampled by the sensor so as to maintain the desired level of environmental air quality. The environmental air quality is sampled by the sensor so that in response to a decrease in the desired environmental air quality, the quantity of air treatment material distributed may be increased. If the predetermined level of environmental air quality is not satisfactory, i.e. the sensor detects a malodor, the sensor communicates with the timing control circuit to provide an increase in air treatment. Specifically, the sensor detects a malodor and sends an indicator signal to the timing control circuit. The timing control circuit then actuates the delivery system accordingly. This action continues until the sensor determines the environmental air quality has returned to the desired level of environmental air quality or the timing control circuit has determined that a significant enough period of increased air treatment has occurred. The sensor determines the desired level of environmental air quality by monitoring the air quality and sending indicator signals to the timing control circuit until the desired level is reached.

During operation of a timed treatment, the duration and intervening time are set according to the selection mechanism and dispense air treatment accordingly. It should be understood that the air treatment system dispenses air treatment according to timed intervals or in response to detection of a malodor.

Further, if the timing control circuit determines a significant period of increased air treatment, the timing control circuit disengages the sensor for an operational time period via the feedback loop. After the time period, the sensor re-engages and once again begins sampling. Additionally, the sensor communicates with the timing control circuit through the feedback loop to determine when the air treatment delivery system has been actuated and, thus, allows the sensor sufficient time to be either off (if the consumer is sensitive to initiating the air treatment system) or to correct the baseline of the sensor response to account for the air treatment system.

In an alternative embodiment, an air-freshening system is shown that combines a continuous dispensing device with a sensing dispensing device. The continuous dispensing device is a plug-in scented oil warmer/dispenser while the sensing dispensing device is similar to the air treatment device described above. The scented oil warmer portion of the device will provide a continuous fragrance, while the sensing air treatment device will be automatically actuated, as described above, when the sensor detects a malodor, providing a large boost of fragrance or an odor neutralizer via an aerosol dispersed from the air treatment device to remove the malodor. Since the aerosol is automatically actuated and is controlled by the odor sensor, there is no active involvement by the user in this device.

It should be understood that the foregoing description is of exemplary embodiments of the invention only, and that the invention is not limited to the specific forms shown. Various modifications may be made in the design and arrangement of the elements set forth herein without departing from the scope of the invention.

What is claimed is:

1. An air treatment device for dispensing an air treatment when needed comprising:
a housing;
a sensor configured to detect a malodor;
a timing control circuit coupled with said sensor, wherein said timing control circuit is configured for both of a timed treatment and a sensor activated treatment;
a selection mechanism for selecting said timed treatment or said sensor activated treatment; and
a delivery system coupled with said timing control circuit.

2. A device as in claim 1 wherein said sensor is a metal oxide sensor.

3. An air treatment device for dispensing an air treatment comprising:
a housing;
a sensor;
a timing control circuit coupled with said sensor; and
a delivery system coupled with said timing control circuit.

4. A device as in claim 3 wherein said timing control circuit is configured for at least one of a timed treatment and a sensor activated treatment.

5. A device as in claim 3 wherein said sensor is configured to detect the intensity of either or both of a malodor and said air treatment.

6. A device as in claim 3 wherein said sensor is configured to detect the presence of either or both of a malodor and said air treatment.

7. A device as in claim 3 wherein said sensor is configured to distinguish between a plurality of malodors.

8. A method for dispensing an air treatment when needed comprising:
detecting a malodor with a sensor;
said sensor communicating with a timing control circuit;
said timing control circuit communicating with a delivery system; and
said delivery system dispensing said air treatment.

9. A method as in claim 8, wherein said step of said timing control circuit communicating with a delivery system further comprises said timing control circuit disengaging said sensor for an operational time period via a feedback circuit.

10. A method as in claim 9, wherein said step of said timing control circuit disengaging said sensor for an operational time period via a feedback circuit further comprises said timing control circuit disengaging said sensor if said timing control circuit determines a significant time period of increased air treatment.

11. A method as in claim 9, wherein said step of said timing control circuit disengaging said sensor for an operational time period via a feedback circuit further comprises said timing control circuit disengaging said sensor if said sensor determines a significantly increased air treatment intensity.

12. A method as in claim 8, wherein said step of said timing control circuit communicating with a delivery system further comprises said timing control circuit providing said delivery system with interval timed programming wherein said air treatment is dispensed in predetermined intervals.
